(19) United States
(12) Patent Application Publication
(10) Pub. No.: US 2017/0274175 A1
(43) Pub. Date: Sep. 28, 2017

(54) CATHETER PACKAGE A SYSTEM FOR CATHETER CONTAINMENT

(71) Applicant: Adrian Brown, Charlotte, NC (US)

(72) Inventor: Adrian Brown, Charlotte, NC (US)

(21) Appl. No.: 15/080,754

(22) Filed: Mar. 25, 2016

(51) Int. Cl. A61M 25/00

(52) U.S. Cl. CPC A61M 25/002

(57) ABSTRACT

A system for catheter containment and application that includes: a catheter package, where said catheter package is adapted to contain residue fluids; a catheter within the catheter package, where the catheter enters a first end and extends through the catheter package; a mid section of the catheter package; a distal end of the catheter package, where a funnel receives fluid from the catheter at the distal end; and lubrication within the catheter package. The system for catheter containment may further includes two sanitizer packs attached to the first end of the catheter package with one sanitizer pack on each side and a contoured mid section. Preferably, the lubrication is provided at the first end of the catheter package.

### BACKGROUND OF THE INVENTION

Field of Invention

The present invention relates to a system for catheter containment and application, which includes a unique package.

Description of Related Art

Many individuals must use a urine catheter due to medical complications related to the expelling of urine. The use of a catheter can sometimes create problems for a user. The catheter essentially is a vent tube, is inserted into a body cavity duct and in particular is used for draining urine from the urinary bladder. As such, use of the catheter can be painful for a patient therefore it is always advantageous to have proper lubrication when inserting the catheter. Further many catheters failed to improve lubrication so therefore making it difficult or necessary to always have lubrication handy when inserting a catheter. Further, the catheter upon removal tends to release residual fluids from the tubing that must be contained during the change out of the catheter. As a result, it would be advantageous to have a device that addressed these two particular issues related to catheter use.

### SUMMARY OF THE INVENTION

The present invention relates to a system for catheter containment and application that includes: a catheter package, where said catheter package is adapted to contain residue fluids and includes a sticky label to seal the package after use; a catheter within the catheter package, where the catheter enters a first end and extends through the catheter package; a mid section of the catheter package; a distal end of the catheter package, where a funnel receives fluid from the catheter at the distal end; and lubrication within the catheter package. The system for catheter containment may further includes two sanitizer packs attached to the first end of the catheter package with one sanitizer pack on each side and a contoured mid section. Preferably, the lubrication is provided at the first end of the catheter package.

### BRIEF DESCRIPTION OF DRAWINGS

FIG. 1 depicts a catheter packaging system in accordance with the present invention.

### DETAILED DESCRIPTION

The present invention relates to a catheter packaging system that provides particular features to assist in the insertion and removal of a urine catheter. The present invention includes a package that surrounds the tip of a catheter and the package includes a sticky label to prevent any spillage of urine after use. The package extends along the length of the catheter tubing at a pre-set distance. At the distal end of the package, the catheter tip lies while the tubing extends through the package at the opposite end or a first end of the catheter package. Lubrication is provided within the package to allow easy access for use during the insertion of the catheter. The catheter package is adapted to accept fluids and preferably can hold a capacity up to eight to ten ounces of fluids to easily catch residue fluid in the tubing. Further, the packaging may also include two sanitary packs attached to the outer surface thereof, preferably a single sanitary pack on each side of the packaging. The sanitary packs may each include a sanitizer that allows a user to quickly sanitize their hands prior to and after use of the catheter. As a result, the present invention provides a complete package to address issues related to the use of a catheter.

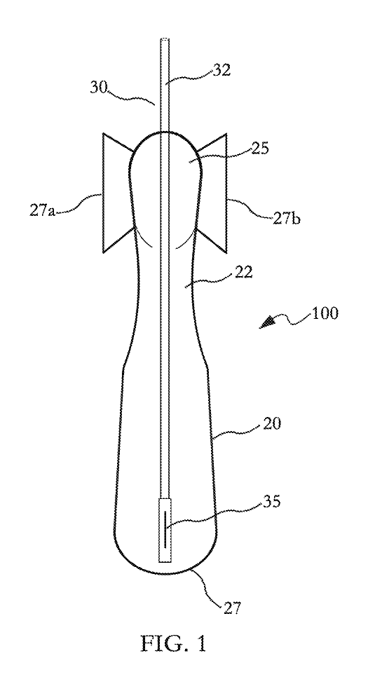

In reference to FIG. 1, a system for catheter containment 100 is depicted. The system includes a catheter package 20 that contains catheter 30 therein. The catheter package 20 includes a sticky label to seal the package 20 after use. As shown, catheter 30 extends through to a distal end 27 of the package 20. A funnel 32 is shown within package 20. At the opposite or first end of package 20 is lubrication 25 within the package 20. This lubrication 25 is used to assist the user and the insertion application of a catheter tip 32 into a patient. Preferably, hand sanitizer packs 27a, 27b is provided on the outer surface of the package 20 near the first end thereof. Further, the package 20 preferably has a contoured middle section 22 where the inner volume of the package 20 is adapted to receive fluids from catheter 30 upon removal. The fluid enters the package 20 through funnel 35 near the distal end 27. This configuration enables a user to contain fluids that normally release when the catheter is removed. The system for catheter containment therefore provides a complete and effective package for the containment and use of a catheter. The instant invention has been shown and described in what it considers to be the most practical and preferred embodiments. It is recognized, however, that departures may be made there from within the scope of the invention and that obvious modifications will occur to a person skilled in the art.

What is claimed is:

1. A system for catheter containment and application comprising:
a. a catheter package, where said catheter package is adapted to contain residue fluids and includes a sticky label to seal the package after use;
b. a catheter within the catheter package, where the catheter enters a first end and extends through the catheter package;
c. a mid section of the catheter package;
d. a distal end of the catheter package, where a funnel receives fluid from the catheter at the distal end; and
e. lubrication within the catheter package.

2. The system for catheter containment according to claim 1, further including two sanitizer packs attached to the first end of the catheter package with one sanitizer pack on each side.

3. The system for catheter containment according to claim 1, further including a contoured mid section.

4. The system for catheter containment according to claim 1, where the lubrication is provided at the first end of the catheter package.
